(19) United States
(12) Patent Application Publication
(10) Pub. No.: US 2010/0028977 A1
(43) Pub. Date: Feb. 4, 2010

(54) ENCLOSED PHOTOBIOREACTORS WITH ADAPTIVE INTERNAL ILLUMINATION FOR THE CULTIVATION OF ALGAE

(75) Inventors: K.Y. Simon Ng, West Bloomfield, MI (US); Nadia Abunasser, Canton, MI (US); Mario E. Danton Garcia Perez, Detroit, MI (US); Steven O. Salley, Grosse Pointe Park, MI (US)

Correspondence Address: BRINKS HOFER GILSON & LIONE, P.O. BOX 10395, CHICAGO, IL, 60610, US

(73) Assignee: WAYNE STATE UNIVERSITY, Detroit, MI (US)

(21) Appl. No.: 12/512,574

(22) Filed: Jul. 30, 2009

Related U.S. Application Data
(60) Provisional application No. 61/084,851, filed on Jul. 30, 2008.

(51) Int. Cl. C12N 1/12; C12M 1/00

(52) U.S. Cl. 435/257.1; 435/292.1

(57) ABSTRACT

The present invention provides an enclosed, internally-lighted bioreactor apparatus, methods for growing photosynthetic microorganisms in the enclosed, internally lighted bioreactor and methods for enhancing growth of a photosynthetic microorganism culture in the enclosed internally-lighted bioreactor.

### CROSS-REFERENCE TO RELATED APPLICATIONS

This application claims the benefit of U.S. Provisional Application Ser. No. 61/084,851 filed on Jul. 30, 2008, entitled “Enclosed Photobioreactors for the Cultivation of Algae with Adaptive Internal Illumination,” the entire contents of which are incorporated herein by reference.

### FEDERALLY SPONSORED RESEARCH AND DEVELOPMENT

This invention was made with government support under Contract No. WSU #446920 awarded by the U.S. Department of Energy/Next Energy. The U.S. government may retain certain rights to the invention.

### FIELD OF THE INVENTION

The present invention relates to processes and systems for the growth of algal cultures in closed photobioreactors using internal lighting. This invention may be designed to grow algae on a large scale to be used as a feedstock for the biofuels industry.

### SUMMARY

There is a need for an alternative non-food crop feedstock for the biofuels industry. Photosynthetic organisms can be used to produce oil that can be converted into biodiesel. In photosynthesis, light, water, and carbon dioxide (CO₂) are converted to carbohydrates, lipids (oils), protein, and oxygen. These reactions can be carried out by chloroplasts and chlorophyll in an algae organism and are thus termed “photobioreactions”.

Algal oil retrieved from a photobioreactor can be processed into biodiesel. Biodiesel engines are often more efficient than gasoline engines. The overall process for producing biofuel from algae typically involves harvesting algae grown in a bioreactor, filtering and drying the harvested algae, and extracting the algal oil. It is the extracted algal oil that is converted to biofuel.

The main reaction for converting various oil to biodiesel is called transesterification. The transesterification process reacts an alcohol (like methanol) with triglyceride oils, forming fatty acid alkyl esters (biodiesel) and glycerin. The reaction requires heat and a strong base catalyst, such as sodium hydroxide or potassium hydroxide.

The one hurdle in the way of using algal oil as the primary alternative feedstock, is the difficulty in growing the algae efficiently, economically, rapidly, and in large enough quantities. Most algal cultivation processes utilize external light sources (i.e. the sun) to illuminate the culture in an open reactor. An example of this is the so-called “open pond” algal bioreactor. Open pond bioreactors have many disadvantages, including contamination and unavailability in areas with cold climates, to name just two. Moreover, reliance on external light limits the growth of the algae when the algal cell concentration is high, since very little light can penetrate into the interior of the open reactor, or open pond reactor, when the algal cell concentration is high.

While internally-lighted, closed bioreactors have advantages over externally-lighted bioreactors, there is still a need for improvement in the area of efficiency, economy, and yield of these bioreactors with respect to the production of algal oil for biofuel. The invention described herein provides a closed system that overcomes the problems associated with known photobioreactors and provides the needed improvements discussed above.

Accordingly, Applicants have developed new photobioreactor designs having improved productivity, thereby reducing the cost, thus making algae a feasible alternative feedstock. Embodiments of the present invention provide, among other improvements, higher yield and faster algal production. The bioreactors and processes of the present invention significantly improve light distribution in the photobioreactors and therefore increase the growth rate of the algae. Further, operating conditions such as for example pH or temperature, are relatively easier to control in the internally illuminated, closed photobioreactors of the present invention.

In one embodiment, the present invention provides an enclosed, internally-lighted bioreactor apparatus comprising a main having an inlet and an outlet, the main being in fluid communication with a source of carbon dioxide-containing gas; an at least one tube extending from the main, the tube comprising walls having a first opening and a second opening defining a chamber, the chamber being configured for containing a culture comprising a liquid and photosynthetic microorganisms, the first opening being formed so that the tube is in fluid communication with the main to allow carbon dioxide-containing gas to flow therethrough for mixing the culture inside the tube and for absorption by the photosynthetic microorganisms; a cover disposed within the walls of the tube to close the chamber; and a strip of light-emitting or light-transmitting material comprising a first portion and a second portion extending from the first portion, the first portion extending through the second opening for receiving light, the second portion disposed in the chamber for emitting light to the culture contained inside the tube for promoting photosynthesis.

In a further embodiment, the present invention provides an enclosed, internally-lighted bioreactor apparatus comprising a container having sidewalls and a floor defining a chamber for housing a culture comprising a liquid and photosynthetic microorganisms, the container having an inlet formed therethrough and in fluid communication with a source of carbon dioxide-containing gas for absorption by the photosynthetic microorganisms a rotatable carousel disposed on the sidewalls to enclose the chamber, the rotatable carousel being variably rotatable about an axis of rotation within the chamber an at least one strip of light-emitting or light-transmitting material, the at least one strip comprising a first portion and a second portion extending from the first portion, the first portion attached to the carousel to receive light and to rotate the strip about the axis of rotation when the carousel rotates, the second portion being disposed in the culture to distribute light within the culture and move about the axis when the carousel rotates.

In a further embodiment, the present invention provides a method for growing photosynthetic microorganisms comprising: introducing carbon dioxide to a culture comprising water, medium, and photosynthetic microorganisms in a covered container; and moving a light-transmitting or light-emitting material through the culture to distribute light to the microorganisms for enhanced growth thereof.

In yet a further embodiment, the present invention provides a method for enhancing the growth of a photosynthetic microorganism comprising: introducing carbon dioxide to an enclosed culture comprising water and photosynthetic microorganisms; moving a light-transmitting or light-emitting material through the culture to distribute light to the microorganisms; monitoring a representative value of the culture, the representative value including one of: pH, viscosity, carousel rotation rate, turbidity, temperature, and dissolved oxygen of the culture; comparing the representative value with a predetermined optimal value to define a comparison; and adjusting one of: pH, viscosity, carousel rotation rate, turbidity, temperature, and dissolved oxygen of the culture based on the comparison.

### BRIEF DESCRIPTION OF THE DRAWINGS

FIG. 1 is a top view of a bioreactor in accordance with one embodiment of the present invention.

FIG. 2 is a cross-sectional view of the bioreactor of FIG. 1 taken along line 2-2.

FIG. 3 is a schematic view of an overall process to produce biodiesel, implementing an aspect of the present invention.

FIG. 4 is a graph generally showing algal growth over time.

FIG. 5a is a top view of a bioreactor in accordance with a further embodiment of the present invention.

FIG. 5b is an enlarged view of a portion 5b of the bioreactor in FIG. 5a.

FIG. 6 is a side view of a cover in accordance with an embodiment of the present invention.

FIG. 7a is a side view of the bioreactor of FIG. 5a.

FIG. 7b is a perspective layout view of the internal air system.

FIG. 7c is a partial perspective view of the bioreactor.

### DETAILED DESCRIPTION

As used herein, the following terms have the meanings given: “feedback control mechanism” means the computer plus various sensors; “medium” means food; “cells” means algal cells; “concentration” means density of algal cells; and “mixture” or “culture” means algae plus medium plus water.

The bioreactors and processes of the present invention differ from existing processes because of the illumination strategy and the adaptive feedback control of the process variables to optimize the growth kinetics. Unlike most of the systems proposed to date, the primary lighting is inside the reactor. The photobioreactors of the present invention do not require direct sunlight for illumination, but instead use light that is collected via solar collectors or lights that are powered using solar batteries. Thus, there is no variation in the light intensity to which the algal culture is exposed throughout the day. Moreover, as discussed below, the adaptive feedback control system of the present invention provides and maintains the optimal operating conditions, including light distribution, for enhancing algal growth. Maintaining these optimal operating conditions increases the productivity of the reactor.

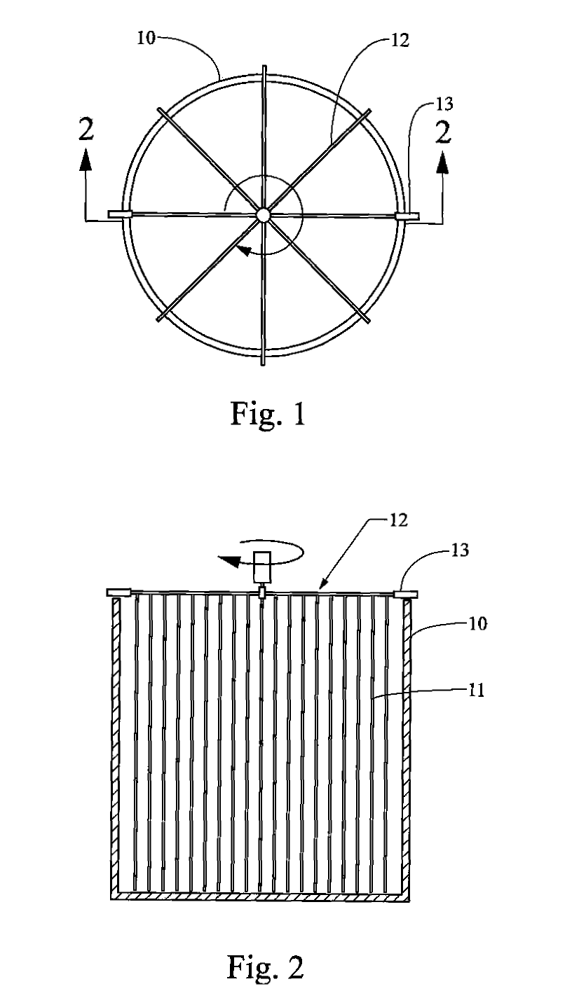

With reference to FIGS. 1 and 2, the bioreactor 10 comprises at least one strip of light-emitting or light-transmitting material 11, preferably a plurality of light-emitting or light-transmitting strips. The second portions of the plurality of strips are spaced apart substantially axially and the carousel 12 is capable of variable rotation about an axis of rotation within the chamber 10. In a further embodiment, at least one distributing arm 13 is mounted on the carousel, the first portion of the at least one light-emitting or light-transmitting strip being attached to the at least one distributing arm. In a further embodiment, the at least one sensor is attached to the distributing arm and rotates with the carousel.

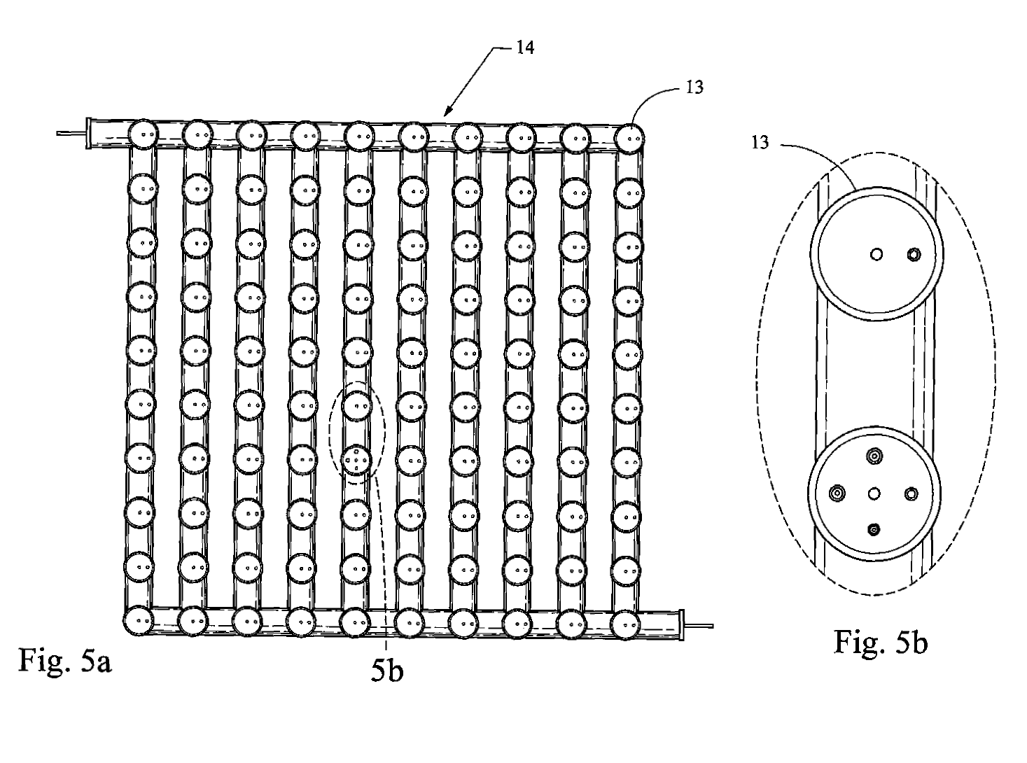

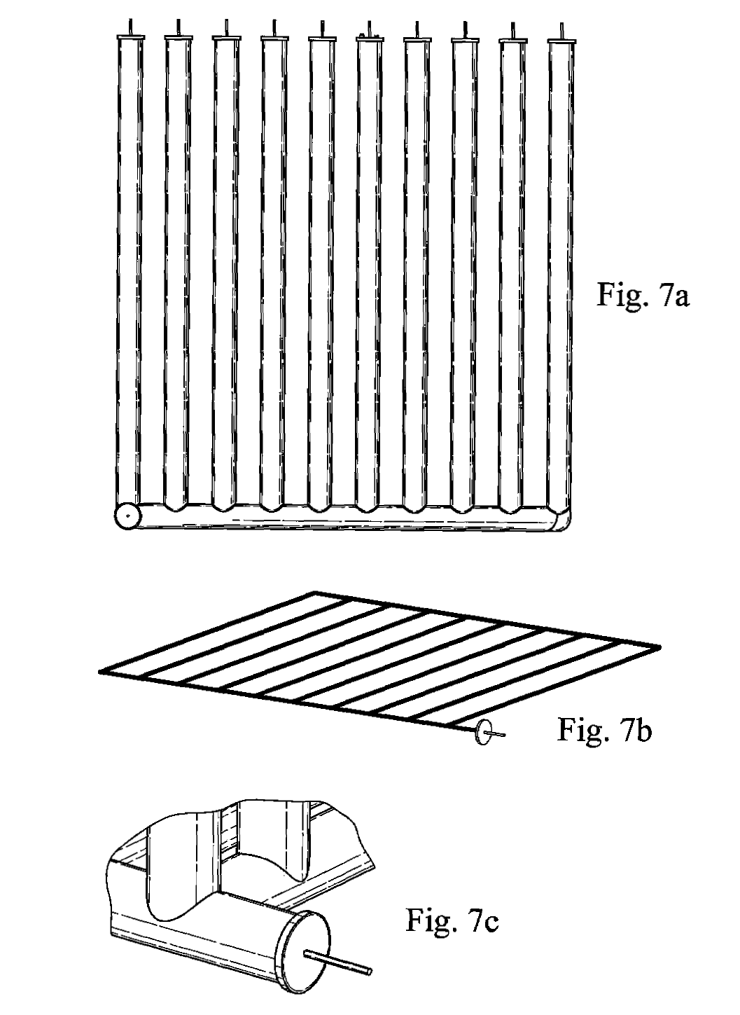

With reference to FIGS. 5a-7c, in one embodiment, the at least one tube 13 is a plurality of tubes.

In a further embodiment, the bioreactor comprises a mechanism that collects light configured to receive light for light transmission through the at least one light-emitting or light-transmitting strip, 11, and transmitting or emitting light to the culture. In an embodiment, the means for collection light is a solar panel. In another embodiment, the means for collecting light is a solar powered battery. In a further embodiment, the at least one strip comprises an optical fiber disposed within the strip and extending therethrough for transmitting light from the means for collecting light to the culture for photosynthesis. In another embodiment, the strip further comprises at least one light emitting diode disposed thereon for emitting light from the means for collection light to the culture for photosynthesis.

In an embodiment of the present invention, the bioreactor further comprises an adaptive feedback control system for growth enhancement of the photosynthetic microorganism culture. In one embodiment, the adaptive feedback control system further comprises a computer and at least one of: a pH sensor, a viscosity sensor, an optical sensor for monitoring turbidity, a temperature sensor, a rotation rate sensor, and a dissolved oxygen sensor.

Further provided is a method for growing photosynthetic organisms comprising monitoring a representative value of the culture, the representative value including one of: pH, viscosity, carousel rotation rate, turbidity, temperature, and dissolved oxygen of the culture; comparing the representative value with a predetermined optimal value to define a comparison; and adjusting one of: pH, viscosity, carousel rotation rate, turbidity, temperature, and dissolved oxygen of the culture based on the comparison. In a further embodiment, the step of adjusting the pH comprises adjusting the mass flow rate of the carbon dioxide-containing gas mixture to control photosynthesis. In yet a further embodiment, the step of adjusting turbidity includes reducing turbidity by removing a fraction of the culture and replacing the fraction with fresh medium to enhance growth of the microorganism.

In an embodiment of the invention, the source of gas comprises exhaust gas from combustion of fossil fuel.

In a preferred embodiment of the bioreactor of present invention the liquid is water and the microorganism is algae.

Exemplary photobioreactor designs according to the present invention have a footprint of around 100 square feet. In one embodiment, as shown in FIGS. 5a-7c, the photobioreactor design is a modular, three-dimensional design containing about 100 vertical tubes, 14 (“tubular bioreactor”), wherein each tube measures about ten feet in length with an approximate 0.5 feet diameter. In the tubular bioreactor, vertical transparent tubes can be oriented such that they are evenly spaced and each tube is exposed to several contingence fluorescent lights. The tubes can be constructed of various algae-compatible materials that are chemical resistant and capable of withstanding typical sterilization processes needed for algal growth, for example, borosilicate glass and similar materials.

In a further embodiment, each tube is fitted with a sparger at the bottom for introducing CO₂ to the tubes. In this case, the CO₂ will be the main source for mixing the culture inside the tube(s). The source of the CO₂ gas can be the waste gas stream of a power plant, thereby providing sequestration of CO₂. Optionally, the waste heat generated can be used in the colder months to heat the reactor.

Several lighting systems can be incorporated in the bioreactors of the present invention, including submersible fiber optics, light-emitting diodes, LED strips and fluorescent lights. Any LED strips known in the art can be adapted for use in the tubular bioreactor. In the case of the submersible LEDs, the design includes the use of solar powered batteries to supply the electricity. This is feasible due to the low voltage needed and high efficiency of the LEDs. In the case of fiber optics, solar collectors with UV filters may be used to bring natural light to the reactor. Nonetheless, the primary lighting system, for example, fiber optics or LEDs, will be submerged inside the tubes. This will provide more efficient distribution and control of the light and light intensity. Florescent lights, external or internal to the photobioreactor, can be used as a back-up system.

In order to fully understand and control the photobioreactor, the present invention further provides an automated data acquisition system which includes pH probes, dissolved oxygen probes, temperature sensors, turbidity sensors, mass flow controllers and a computer data acquisition system for monitoring and controlling the various operating parameters. Any submergible sensor known in the art can be adapted for use with the bioreactors of the present invention. These various probes and sensors are known to those skilled in the art.

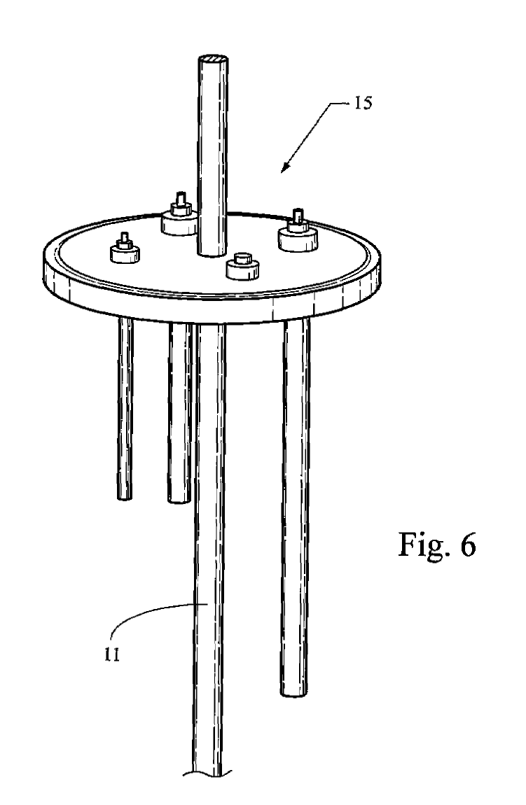

In the tubular bioreactor, the sensors may, for example, be disposed in the tube and extend through the second opening of the tube and through an opening on the cover 15 as shown in FIG. 6.

In another embodiment, as shown in FIGS. 1 and 2, the photobioreactor design is a tank reactor, e.g., having a height of approximately ten feet and having a diameter of approximately ten feet. However, any type of tank reactor known in the art can be adapted for use as the bioreactor. The bioreactor can be constructed from any material suitable for use in constructing standard water tanks, for example, stainless steel optionally fitted with a glass liner. An advantage of this design is the high volume to footprint ratio.

In a further embodiment of the present invention, the tank photobioreactor is fitted with a rotatable carousel of submersible lighting. In a further embodiment, the carousel of lighting is an array of flexible, submersible LED strips, capable of gently rotating inside the reactor. Any LED strips known in the art can be adapted for use in the tank bioreactor of the present invention. In this embodiment, larger depths and widths are possible, and thus a higher areal productivity, as a direct result of using rotating submersible lighting.

In one embodiment, the strips are about ten feet in length and up to one feet in diameter. In a further embodiment, a first portion of each of the LED strips is attached to the carousel. In a further embodiment, the first portion of each of the LED strips is attached to a distributing arm mounted on the carousel. Any mounting means known in the art can be adapted for use with the tank bioreactor, including for example, screws, staples and clips. Any submergible sensor known in the art can be adapted for use with the tank reactor of the present invention. In a further embodiment, each of the sensors is attached to the distributing arm of the carousel. Accordingly, each of the sensors rotates as the carousel rotates, thereby providing feedback for the entire chamber of the bioreactor.

Rotating the carousel, and the flexible strips thereon, through the water, has several advantages. First, rotating the light strips through the water achieves greater uniformity of light intensity throughout the tank. Further, the strips can be gently rotated, which is beneficial to enhancing algal growth.

Generally, all algae comprise the following compounds, in varying proportions: Proteins, Carbohydrates, Lipids (or Oils or Fatty acids) and Nucleic Acids. An exemplary indication of those proportions is shown in Table 1.

TABLE 1
Chemical Compositions of Algae Expressed
on A Dry Matter Basis (%)

| | | Carbo- | | Nucleic |
|---|---|---|---|---|
| Strain | Protein | hydrates | Lipids | Acid |
| Scenedesmus obliquus | 50-56 | 10-17 | 12-14 | 3-6 |
| Scenedesmus quadricauda | 47 | — | 1.9 | — |
| Scenedesmus dimorphus | 8-18 | 21-52 | 16-40 | — |
| Chlamydomonas rheinhardii | 48 | 17 | 21 | — |
| Chlorella vulgaris | 51-58 | 12-17 | 14-22 | 4-5 |
| Chlorella pyrenoidosa | 57 | 26 | 2 | — |
| Spirogyra sp. | 6-20 | 33-64 | 11-21 | — |
| Dunaliella bioculata | 49 | 4 | 8 | — |
| Dunaliella salina | 57 | 32 | 6 | — |
| Euglena gracilis | 39-61 | 14-18 | 14-20 | — |
| Prymnesium parvum | 28-45 | 25-33 | 22-38 | 1-2 |
| Tetraselmis maculate | 52 | 15 | 3 | — |
| Porphyridium cruentum | 28-39 | 40-57 | 9-14 | — |
| Spirulina platensis | 46-63 | 8-14 | 4-9 | 2-5 |
| Spirulina maxima | 60-71 | 13-16 | 6-7 | 3-4.5 |
| Synechoccus sp. | 63 | 15 | 11 | 5 |
| Anabaena cylindrical | 43-56 | 25-30 | 4-7 | — |

Source: Becker, (1994)

While the percentages vary with the type of algae, there are algae types that are comprised of up to 40% of their overall mass by fatty acids or oil. It is this fatty acid or oil that can be extracted and converted into biodiesel by techniques identifiable by a skilled person. Algal oil is generally very high in unsaturated fatty acids, such as Arachidonic acid (AA), Eicospentaenoic acid (EPA), Docasahexaenoic acid (DHA), Gamma-linolenic acid (GLA), and Linoleic acid (LA). Depending on the exact strain of algae used, and precise bioreactor design in accordance with the present invention outlined herein, productivity of algal oil can be up to twenty gallons per month.

Another feature of the present invention involves the controlled addition of the reactant gas, e.g. CO₂, to the algal culture in the closed photobioreactor. Algal cultures typically obtain their carbon from a gas, e.g. CO₂, which is bubbled through the algal culture, establishing the following equilibria:

CO₂+H₂O [symbol] H₂CO₃ [symbol] H⁺+HCO₃ [symbol] H⁺+CO₃⁻²

Further, the equation for photosynthesis is as follows:

6CO₂+6H₂O+Energy→C₆H₁₂O₆+6O₂

Thus, a photosynthetic algal culture will consume CO₂, resulting in the increased alkalization of the solution.

As the concentration of CO₂ in the mixture goes up, pH goes down. Conversely, as the concentration of CO₂ in the mixture goes down, pH goes up. Either extreme, too high or too low of pH, is detrimental to the algae. Accordingly, in order to enhance algae growth, it is advantageous to maintain the pH at an optimal value. Thus, it is advantageous to monitor pH and adjust it accordingly. The controlled addition of CO₂ to the mixture may be used to adjust pH to a predetermined optimal value. Optimal values for pH for algae were predetermined in lab-scale (bottle reactor)(s) quantities using known scientific methods. Of course, precise optimum pH value will vary depending on exact strain of algae. These values can be determined by one of skill in the art.

As the algal cell concentration goes up, turbidity goes up. In order to enhance algae growth, it is advantageous to maintain the concentration, as much as possible, in the exponential growth phase (See FIG. 4). In order to maximize growth of the algae, it is advantageous to monitor turbidity. Thus, in one embodiment, the sensor is an optical sensor to measure turbidity. If the turbidity becomes too high as compared to the optimum value, then, some algae can be removed from the culture and replaced with fresh medium.

As the algal cell concentration increases, viscosity will also increase. As the viscosity increases, the rotation rate of the carousel will be automatically adjusted to a higher rotation rate to maintain the uniform distribution of light and intensity. The reverse is also true, that is, if the turbidity or viscosity goes down, the carousel will be automatically adjusted to a lower rotation rate. Any motor known in the art, for example a Stepp motor, can be adapted for use with the bioreactors of the present invention.

Measurements for all of the various sensors described and exemplified above, are fed into a computer. The computer will control light intensity and rate of rotation. Any computer known in the art can be adapted for use with the reactors of the present invention.

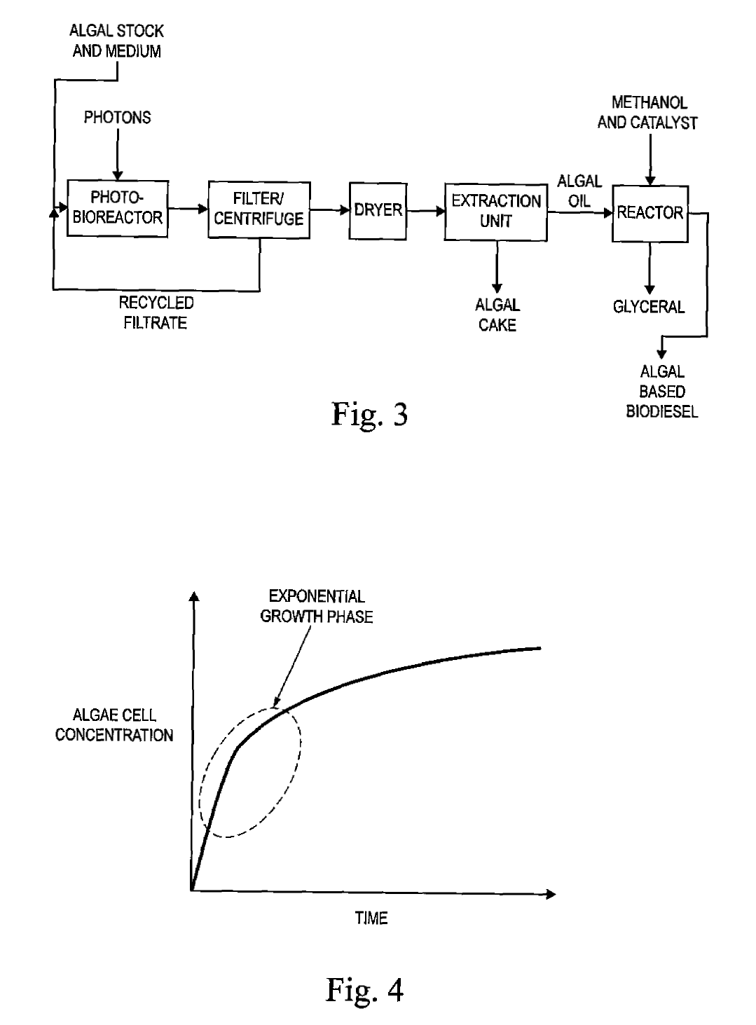

FIG. 3 depicts a system or process in which an embodiment of the bioreactor is implemented to produce biodiesel. As shown, the algal stock and medium along with photons from a light source or other source are introduced to the bioreactor. The output is then introduced to a filter/centrifuge unit and then dried in a drying unit. As shown, recycled filtrate is directed back to the algal stock and medium feed stream. The output of the drying unit is directed to an extraction unit where an algal cake stream directs algal cake out to a preferably separate processing system (not shown) and an algal oil stream containing algal oil leads to a reactor. At the reactor, methanol and catalyst are introduced to react with the algal oil to provide at least two streams: a glycerol output and an algal based biodiesel output stream.

While various embodiments for carrying out the invention have been described in detail, those familiar with the art to which this invention relates will recognize various alternative designs and embodiments for practicing the invention as defined by the following claims.

1. An enclosed, internally-lighted bioreactor apparatus comprising:
a main having an inlet and an outlet, the main being in fluid communication with a source of carbon dioxide-containing gas;
an at least one tube extending from the main, the tube comprising walls having a first opening and a second opening defining a chamber, the chamber being configured for containing a culture comprising a liquid and photosynthetic microorganisms, the first opening being formed so that the tube is in fluid communication with the main to allow carbon dioxide-containing gas to flow therethrough for mixing the culture inside the tube and for absorption by the photosynthetic microorganisms;
a cover disposed within the walls of the tube to close the chamber; and
a strip of light-emitting or light-transmitting material comprising a first portion and a second portion extending from the first portion, the first portion extending through the second opening for receiving light, the second portion disposed in the chamber for emitting light to the culture contained inside the tube for promoting photosynthesis.

2. The bioreactor of claim 1 wherein the at least one tube is a plurality of tubes.

3. The bioreactor of claim 1 wherein the liquid is water and the microorganism is algae.

4. An enclosed, internally-lighted bioreactor apparatus comprising:
a container having sidewalls and a floor defining a chamber for housing a culture comprising a liquid and photosynthetic microorganisms, the container having an inlet formed therethrough and in fluid communication with a source of carbon dioxide-containing gas for absorption by the photosynthetic microorganisms;
a rotatable carousel disposed on the sidewalls to enclose the chamber, the rotatable carousel being variably rotatable about an axis of rotation within the chamber;
an at least one strip of light-emitting or light-transmitting material, the at least one strip comprising a first portion and a second portion extending from the first portion, the first portion attached to the carousel to receive light and to rotate the strip about the axis of rotation when the carousel rotates, the second portion being disposed in the culture to distribute light within the culture and move about the axis when the carousel rotates.

5. The bioreactor of claim 4 further comprising means for collecting light configured to receive light for light transmission through the at least one light-emitting or light-transmitting strip and transmitting or emitting light to the culture.

6. The bioreactor of claim 5 wherein the at least one light-emitting or light transmitting strip further comprises an optical fiber disposed within the strip and extending therethrough for transmitting light from the means for collecting light to the culture for photosynthesis.

7. The bioreactor of claim 5 wherein the at least one light-emitting or light-transmitting strip further comprises at least one light emitting diode disposed thereon for emitting light from the means for collecting light to the culture for photosynthesis.

8. The bioreactor of claim 6 wherein the means for collecting light is a solar panel.

9. The bioreactor of claim 6 wherein the means for collecting light comprises a solar powered battery.

10. The bioreactor of claim 4 wherein the at least one strip of light-emitting or light-transmitting material is a plurality of light-emitting or light-transmitting strips, the second portions of the plurality of strips being spaced apart substantially axially; and
the carousel capable of variable rotation about an axis of rotation within the chamber.

11. The bioreactor of claim 4 wherein the liquid is water and the microorganism is algae.

12. The bioreactor of claim 5 wherein the source of gas comprises exhaust gas from combustion of fossil fuel.

13. The bioreactor of claim 5 further comprising an adaptive feedback control system for growth enhancement of the photosynthetic microorganism culture.

14. The bioreactor of claim 13 wherein the adaptive feedback control system further comprises a computer and at least one of: a pH sensor, a viscosity sensor, an optical sensor for monitoring turbidity, a temperature sensor, a rotation rate sensor, and a dissolved oxygen sensor.

15. The bioreactor of claim 14 further comprising at least one distributing arm mounted on the carousel, the first portion of the at least one light-emitting or light-transmitting strip being attached to the at least one distributing arm.

16. The bioreactor of claim 15 wherein the at least one sensor is attached to the distributing arm and rotates with the carousel.

17. A method for growing photosynthetic microorganisms comprising:
introducing carbon dioxide to a culture comprising water, medium, and photosynthetic microorganisms in a covered container; and
moving a light-transmitting or light-emitting material through the culture to distribute light to the microorganisms for enhanced growth thereof.

18. The method of claim 17 further comprising:
monitoring a representative value of the culture, the representative value including one of: pH, viscosity, carousel rotation rate, turbidity, temperature, and dissolved oxygen of the culture;
comparing the representative value with a predetermined optimal value to define a comparison; and
adjusting one of: pH, viscosity, carousel rotation rate, turbidity, temperature, and dissolved oxygen of the culture based on the comparison.

19. The method of claim 18 wherein the step of adjusting the pH comprises adjusting the mass flow rate of the carbon dioxide-containing gas mixture to control photosynthesis.

20. The method of claim 18 wherein the step of adjusting turbidity includes reducing turbidity by removing a fraction of the culture and replacing the fraction with fresh medium to enhance growth of the microorganism.

21. The method of claim 20 wherein the microorganism is algae.

22. A method for enhancing the growth of a photosynthetic microorganism comprising:
introducing carbon dioxide to an enclosed culture comprising water and photosynthetic microorganisms;
moving a light-transmitting or light-emitting material through the culture to distribute light to the microorganisms;
monitoring a representative value of the culture, the representative value including one of: pH, viscosity, carousel rotation rate, turbidity, temperature, and dissolved oxygen of the culture;
comparing the representative value with a predetermined optimal value to define a comparison; and
adjusting one of: pH, viscosity, carousel rotation rate, turbidity, temperature, and dissolved oxygen of the culture based on the comparison.

23. The method of claim 22 wherein the microorganism is algae.
